(19) United States
(12) Patent Application Publication
(10) Pub. No.: US 2024/0081797 A1
(43) Pub. Date: Mar. 14, 2024

(54) SECURING A GUIDEWIRE DELIVERY CATHETER IN THE CORONARY SINUS USING A MECHANICALLY RELEASING ARM

(71) Applicant: EDWARDS LIFESCIENCES CORPORATION, Irvine, CA (US)

(72) Inventors: Bethany Jo Hall, Newport Beach, CA (US); Cooper Ryan Rickerson, Huntington Beach, CA (US); Jason James Raid Haddad, Corona, CA (US); Steven Charles Coutteau, Ferndale, MI (US)

(21) Appl. No.: 18/513,113

(22) Filed: Nov. 17, 2023

Related U.S. Application Data
(63) Continuation of application No. PCT/US2022/030195, filed on May 20, 2022.
(60) Provisional application No. 63/191,419, filed on May 21, 2021.

(51) Int. Cl. A61B 17/00

(52) U.S. Cl. CPC A61B 17/00234; A61B 2017/00252; A61B 2017/00867

(57) ABSTRACT

Mechanisms are disclosed for securing a catheter in place to facilitate puncturing a hole through a vessel wall. The securing mechanisms include mechanically releasing arms that press against the wall of the vessel. The mechanically releasing arms are advanced out of the catheter. Outside of the catheter, the securing mechanisms angle or curve toward the vessel wall to contact the wall. The more the mechanically releasing arms are advanced, the closer the approach to the wall and the more force placed against the wall to anchor the catheter in place. The mechanically releasing arms include a plurality of wires that angle or curve toward the vessel wall when deployed, one or more wires that coil away from the catheter to contact the vessel wall when deployed, or a stopper arm with a curved endcap that contacts the vessel wall when deployed.

### CROSS-REFERENCE TO RELATED APPLICATION

This application is a continuation of International Patent Application No. PCT/US2022/030195, filed May 20, 2022 and entitled SECURING A GUIDEWIRE DELIVERY CATHETER IN THE CORONARY SINUS USING A MECHANICALLY RELEASING ARM, which claims the benefit of priority of U.S. Prov. App. No. 63/191,419, filed May 21, 2021 and entitled SECURING A GUIDEWIRE DELIVERY CATHETER IN THE CORONARY SINUS USING A MECHANICALLY RELEASING ARM, the complete disclosures of which are hereby incorporated by reference in their entireties for all purposes.

### BACKGROUND

### Field

The present invention relates generally to the field of delivery devices, such as catheters, for medical procedures involving the coronary sinus.

### Description of Related Art

Heart failure is a common and potentially lethal condition affecting humans, with sub-optimal clinical outcomes often resulting in morbidity and/or mortality, despite maximal medical treatment. In particular, “diastolic heart failure” refers to the clinical syndrome of heart failure occurring in the context of preserved left ventricular systolic function (ejection fraction) and in the absence of major valvular disease. This condition is characterized by a stiff left ventricle with decreased compliance and impaired relaxation, which leads to increased end-diastolic pressure.

Symptoms of diastolic heart failure are due, at least in large part, to an elevation in pressure in the left atrium. Elevated left atrial pressure (LAP) is present in several abnormal heart conditions, including heart failure (HF). In addition to diastolic heart failure, a number of other medical conditions, including systolic dysfunction of the left ventricle and valve disease, can lead to elevated pressures in the left atrium. Both heart failure with preserved ejection fraction (HFpEF) and heart failure with reduced ejection fraction (HFrEF) can exhibit elevated LAP.

It may be beneficial to reduce elevated pressure in the left atrium. One way to do this is to shunt blood from the left atrium to the coronary sinus. By creating an opening between the left atrium and the coronary sinus, blood will flow from the higher pressure left atrium to the lower pressure coronary sinus. Examples of methods to shunt blood from the left atrium to the coronary sinus are disclosed in U.S. Pat. No. 9,789,294 entitled “Expandable Cardiac Shunt,” the entire contents of which is incorporated by reference herein.

Using catheter-based instruments, the surgeon creates a puncture hole between the left atrium and the coronary sinus and places an expandable shunt within the puncture hole. To do this, one or more catheters are used to create the puncture hole, to deliver the expandable shunt along a guidewire, and to deploy the expandable shunt in the puncture hole. Once expanded, the shunt defines a blood flow passage that allows blood to flow from the left atrium to the coronary sinus when the LAP is elevated.

### SUMMARY

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Some implementations of the present disclosure relate to a guidewire delivery catheter used to implant a shunt between the coronary sinus and the left atrium. The catheter includes a needle housed within the catheter in a delivery configuration, the needle configured to extend out of the catheter to puncture a vessel wall in a deployment configuration. The catheter includes a needle port through which the needle extends out of the catheter to transition from the delivery configuration to the deployment configuration. The catheter includes a securing mechanism housed within the catheter in a delivery configuration, the securing mechanism comprising a compliant wire configured to be advanced out of the catheter in a deployment configuration so that a distal portion of the wire comes into contact with the vessel wall to provide wall apposition for the needle to puncture the vessel wall. The catheter includes a wire port through which the wire of the securing mechanism extends out of the catheter to transition from the delivery configuration to the deployment configuration.

In some examples, a distal portion of the wire of the securing mechanism is configured to coil upon exiting the wire port.

In some examples, the catheter further includes a second wire port, wherein the securing mechanism further comprises a second wire configured to exit the catheter at the second wire port. In further examples, each of the wires of the securing mechanism is configured to curve away from the catheter in the deployment configuration. In further examples, an azimuth angle between the needle port and each of the two wire ports is at least 90 degrees and less than 180 degrees. In further examples, an azimuth angle between the needle port and each of the two wire ports is at least 120 degrees. In further examples, an azimuth angle (θ1) between the two wire ports is less than or equal to 120 degrees.

In some examples, the securing mechanism further includes a curved endcap at a distal end of the wire. In further examples, the curved endcap is configured to contact the vessel wall in the deployment configuration. In further examples, the curved endcap is configured to cover the wire port in the delivery configuration. In further examples, the curved endcap is configured to follow a contour of the vessel wall.

In some examples, the wire is configured to run along a length of the catheter such that the wire is configured to be advanced by manipulating a component of the securing mechanism from outside of an operative site.

In some examples, the wire comprises a shape memory metal. In further examples, the wire is shape set to curve away from the catheter. In further examples, the wire is shape set to coil outside of the catheter. In further examples, the wire is shape set to exit the catheter at an exit angle. In further examples, a distal end of the catheter is coated with a material to reduce a likelihood of damaging the vessel wall.

In some examples, the wire is configured to be withdrawn to transition from the deployment configuration back to the delivery configuration. In some examples, the wire port is positioned opposite the needle port so that the wire exits the catheter on an opposite side from where the needle exits the catheter. In some examples, wire port is positioned more distally along the catheter relative to the needle port.

### BRIEF DESCRIPTION OF THE DRAWINGS

Various examples are depicted in the accompanying drawings for illustrative purposes and should in no way be interpreted as limiting the scope of the disclosed subject matter. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 illustrates several access pathways for maneuvering guidewires and catheters in and around the heart to deploy shunts.

FIG. 2 depicts one approach method for deploying an expandable shunt, wherein a guidewire is introduced through the subclavian or jugular vein, through the superior vena cava and into the coronary sinus.

FIGS. 3A, 3B, 3C, and 3D are schematic views of steps in making a puncture hole through a wall of the coronary sinus, as seen looking down on a section of the heart with the posterior aspect down.

FIGS. 4A, 4B, 4C, and 4D illustrate a first example securing mechanism.

FIGS. 5A, 5B, and 5C illustrate a second example securing mechanism.

FIGS. 6A, 6B, and 6C illustrate a third example securing mechanism.

### DETAILED DESCRIPTION

The headings provided herein are for convenience only and do not necessarily affect the scope or meaning of the claimed subject matter.

### Overview

Symptoms of diastolic heart failure arise from elevated pressure in the left atrium, or elevated left atrial pressure (LAP). Other heart conditions may manifest elevated LAP as well. To reduce the pressure in the left atrium, a pathway can be created between the left atrium and the coronary sinus. This allows blood to flow from the higher pressure left atrium to the lower pressure coronary sinus. The pathway can be created by puncturing a hole between the left atrium and the coronary sinus. Once the hole has been created, a shunt can be placed in the hole to keep it open.

For example, catheter-based instruments can be used to create the hole and to deliver and deploy a shunt within the puncture hole. The catheter can be referred to as a guidewire delivery catheter (GDC) and can be used to puncture through the coronary sinus into the left atrium and to place a guidewire in the left atrium. To puncture through the tissue, the catheter has wall apposition to get the needle directly against the coronary sinus-left atrium wall. Prior GDCs used an “anchor balloon” (a saline-inflated balloon) to anchor the catheter when creating the puncture hole. The anchor balloon solution has the potential to burst or to slip out of position, which may harm the patient.

Accordingly, described herein are materials and mechanisms for securing the GDC in place to enable the GDC to puncture the wall from the coronary sinus to the left atrium. These materials and mechanisms serve as an alternative to the anchor balloon.

The term “catheter” is used herein according to its broad and ordinary meaning and may include any tube, sheath, steerable sheath, steerable catheters, and/or any other type of elongate tubular delivery device comprising an inner lumen configured to slidably receive instrumentation, such as for positioning within an atrium or coronary sinus, including for example delivery catheters and/or cannulas. In some cases, a securing mechanism may be composed of a shape-memory alloy (e.g., Nitinol) and/or may have a pre-defined shape and/or structure. The securing mechanism may be configured to be shaped and/or compressed to fit into and/or around a catheter. In some cases, a securing mechanism may have an elongated shape in a delivery configuration to extend at least partially along the catheter and to change shape in a deployed configuration to provide wall apposition.

Some examples described herein provide methods and/or systems for securing a guidewire delivery catheter, or other similar delivery device, to provide wall apposition for puncturing a vessel wall. While some examples may be directed to securing a catheter within the coronary sinus to puncture the wall between the coronary sinus and the left atrium, the devices described herein may be applicable to other areas of the body. For example, some devices described herein may advantageously be configured for securing catheters to provide wall apposition for puncturing vessels in vessels other than the coronary sinus.

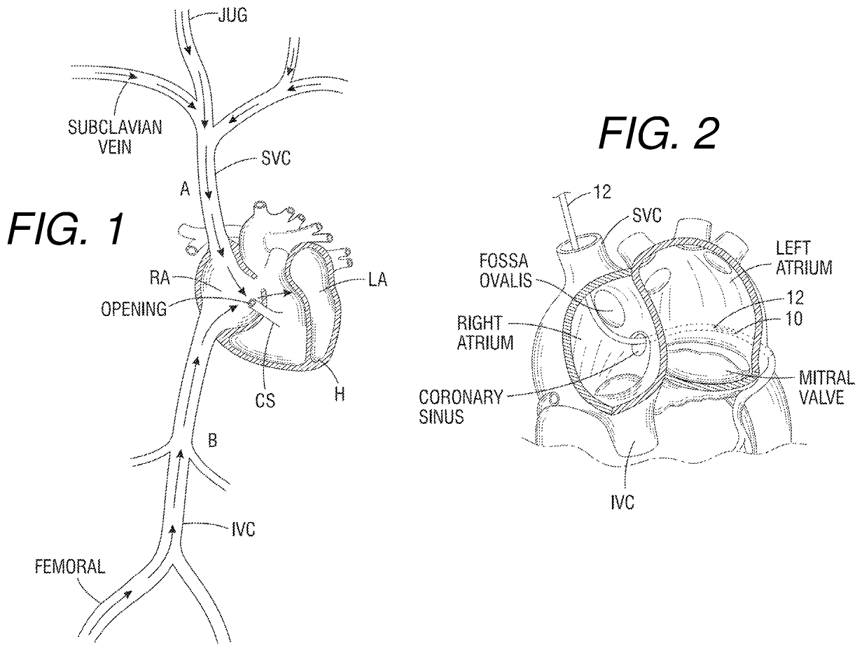

The following includes a general description of a method for delivering a guidewire delivery catheter to a targeted location in the coronary sinus. It is to be understood that the disclosed securing mechanisms can be used in conjunction with such a guidewire delivery catheter in this or similar methods to provide wall apposition for puncturing the coronary sinus. FIG. 1 illustrates several access pathways for maneuvering guidewires and catheters in and around the heart to deploy shunts. For instance, access may be from above via either the subclavian or jugular veins into the superior vena cava (SVC), right atrium (RA) and from there into the coronary sinus (CS). Alternatively, the access path may start in the femoral vein and through the inferior vena cava (IVC) into the heart. Other access routes may also be used, and each typically utilizes a percutaneous incision through which the guidewire and catheter are inserted into the vasculature, normally through a sealed introducer, and from there the physician controls the distal ends of the devices from outside the body.

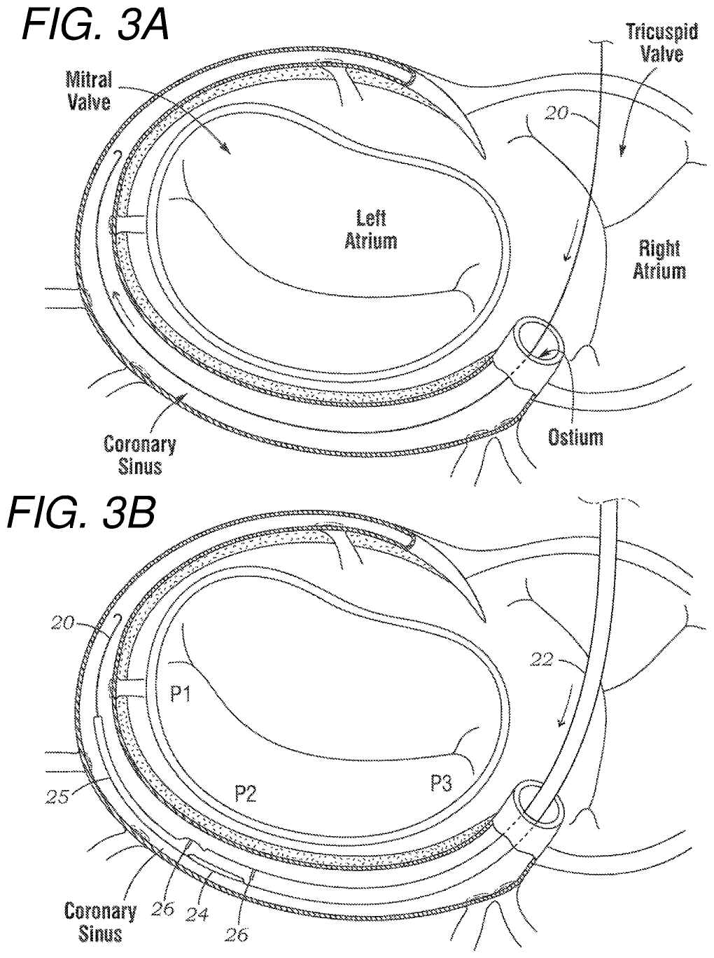

FIG. 2 depicts an example method for deploying an expandable shunt, wherein a guidewire 10 is introduced through the subclavian or jugular vein, through the superior vena cava and into the coronary sinus. Once the guidewire provides a path, an introducer sheath (not shown) may be routed along the guidewire and into the patient's vasculature, typically with the use of a dilator. FIG. 2 shows a deployment catheter 12 extending from the superior vena cava to the coronary sinus of the heart, the deployment catheter 12 having been passed through the introducer sheath which provides a hemostatic valve to prevent blood loss.

In some examples, the deployment catheter 12 may be about 30 cm long, and the guidewire 10 may be somewhat longer for ease of use. In certain examples, the deployment catheter 12 may function to form and to prepare an opening in the wall of the left atrium, and a separate placement or delivery catheter may be used for delivery of an expandable shunt. In various examples, the deployment catheter may be used for both puncture preparation and shunt placement. In the present application, the terms “deployment catheter” or “delivery catheter” are used to represent a catheter or introducer with one or both of these functions.

Since the coronary sinus is largely contiguous around the left atrium, there are a variety of possible acceptable placements for a stent. The site selected for placement of the stent may be made in an area where the tissue of the particular patient is less thick or less dense, as determined beforehand by non-invasive diagnostic means, such as a CT scan or radiographic technique, such as fluoroscopy or intravascular coronary echo (IVUS).

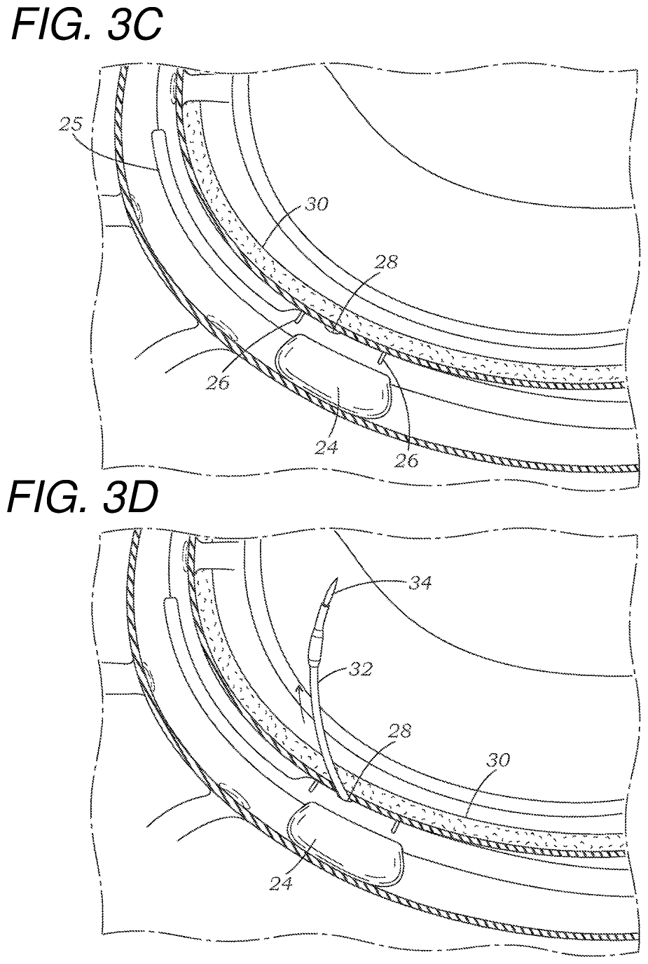

FIGS. 3A-3D are schematic views of steps in making a puncture hole through a wall of the coronary sinus, as seen looking down on a section of the heart with the posterior aspect down. Initially, FIG. 3A shows a guidewire 20 being advanced from the right atrium into the coronary sinus through its ostium or opening. A puncture catheter 22 is then advanced over the guidewire 20, as seen in FIG. 3B. The puncture catheter 22 is introduced into the body through a proximal end of an introducer sheath (not shown). As is customary, an introducer sheath provides access to the particular vascular pathway (e.g., jugular or subclavian vein) and may have a hemostatic valve therein. While holding the introducer sheath at a fixed location, the surgeon manipulates the puncture catheter 22 to the implant site.

In certain implementations, a distal end of the puncture catheter 22 has a slight curvature, with a radially inner and a radially outer side, to conform to the curvature of the coronary sinus. A securing mechanism 24 is exposed along the radially outer side of the catheter 22 adjacent an extreme distal segment 25 that may be thinner than or tapered narrower from the proximal extent of the catheter. Radiopaque markers 26 on the catheter 22 help the surgeon determine the precise advancement distance for desired placement of the securing mechanism 24 within the coronary sinus. In some instances, the radiopaque markers 26 are C-shape bands that flank the proximal and distal ends of the securing mechanism 24.

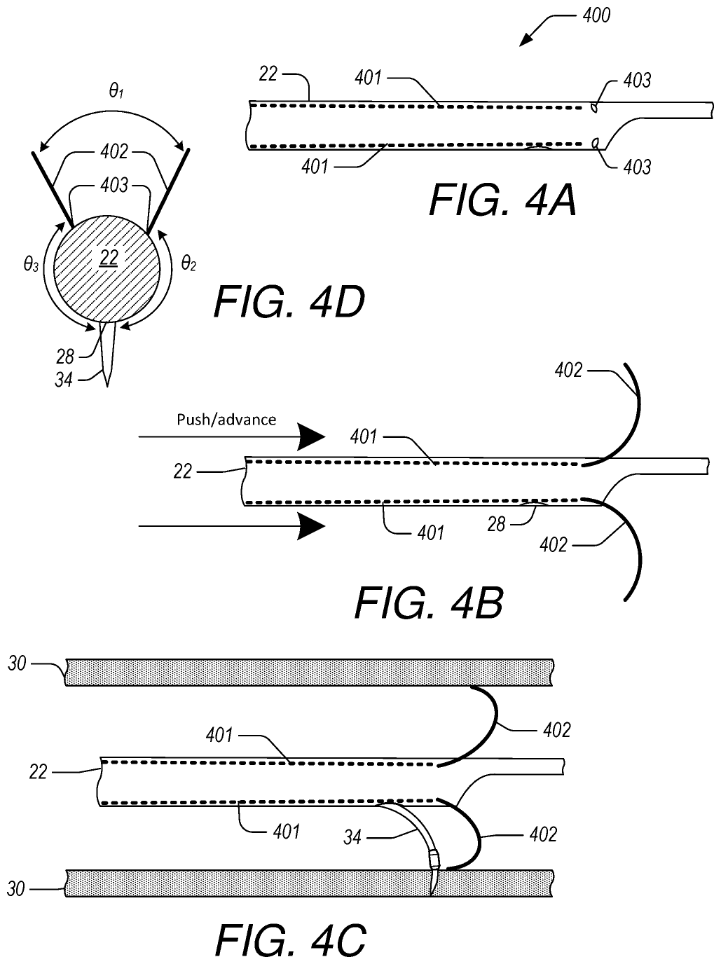

FIG. 3C shows outward deployment of the securing mechanism 24, which is to be considered a generic structure that is replaced by any of the securing mechanisms disclosed herein and described with reference to FIGS. 4A-6C. Deployment of the securing mechanism 24 presses the radially inner curve of the catheter against the luminal wall of the coronary sinus to provide wall apposition. Again, the securing mechanism 24 is located adjacent the distal segment 25 of the puncture catheter 22. In some examples, the securing mechanism 24 extends opposite a needle port 28 formed in the radially inner side wall of the catheter. Consequently, the needle port 28 abuts the luminal wall and faces toward a tissue wall 30 between the coronary sinus and the left atrium. Preferably, guided by visualizing the radiopaque markers 26, the surgeon advances the catheter 22 so that the needle port 28 is located within about 2-4 cm into the coronary ostia. This places the subsequent puncture approximately above the “P2” portion of the posterior leaflet of the mitral valve (when looking at the inflow side of the valve the posterior leaflet has P1-P2-P3 cusps in a CCW direction, as seen in FIG. 3B). The securing mechanism 24 may be centered diametrically across the catheter 22 from the needle port 28, or as shown may be slightly offset in a proximal direction from the needle port 28 to improve leverage.

The curvature at the distal end of the puncture catheter 22 aligns to and “hugs” the anatomy within the coronary sinus and orients the needle port 28 inward, while the securing mechanism 24 holds the catheter 22 in place relative to the coronary sinus. Subsequently, as seen in FIG. 3D, a puncture sheath 32 having a puncture needle 34 with a sharp tip advances along the catheter 22 such that it exits the needle port 28 at an angle from the longitudinal direction of the catheter and punctures through the wall 30 into the left atrium. The puncture sheath 32 has a built-in curvature at the end that “aligns” with the curvature of the anatomy ensuring that the needle 34 is oriented inward toward the left atrium. The securing mechanism 24 provides rigidity to the system and holds the needle port 28 against the wall 30 (e.g., the securing mechanism 24 provides wall apposition). Preferably, the puncture needle 34 has a flattened configuration to form a linear incision and is mounted on the distal end of an elongated wire or flexible rod (not shown) that passes through a lumen of the puncture sheath 32.

### Securing Mechanisms

Described herein are mechanisms for securing a catheter within a vessel to facilitate puncturing a wall of the vessel with a needle that extends from the catheter. The securing mechanisms described herein can be implemented in a catheter, such as the catheter 22 described herein with respect to FIGS. 3A-3D. Additionally, the securing mechanisms described herein are configured to provide the functionality of the generic securing mechanism 24 described herein with respect to FIGS. 3A-3D. In other words, the securing mechanisms described below can be used in place of the securing mechanism 24 and/or to provide the functionality of the securing mechanism 24, as described herein with respect to FIGS. 3A-3D.

The disclosed securing mechanisms are variations of the securing mechanism 24 that is used to secure the guidewire delivery catheter in place to facilitate puncturing the wall between the coronary sinus and the left atrium. The disclosed example securing mechanisms represent an improvement over other securing mechanisms, such as a saline-filled anchor balloon, because there is no risk of balloon burst and there is potential for better catheter securement. If the balloon bursts before the needle is deployed, the needle could deploy into the wrong space potentially causing severe damage to the patient. Moreover, a ruptured balloon could result in an embolism in the patient. The disclosed examples eliminate this risk. Furthermore, the disclosed examples eliminate the need to deflate the balloon, thereby providing a more streamlined puncturing procedure.

The disclosed securing mechanisms include mechanically releasing arms that are actuated to press against the coronary sinus wall to provide apposition for the guidewire delivery catheter, thereby ensuring that the needle punctures properly through the coronary sinus wall and into the left atrial space. The disclosed securing mechanisms comprise a compliant wire that extends outward from the catheter. The wire can be made from a shape memory material such as a nickel titanium alloy (e.g., Nitinol). The securing mechanisms can be activated by pushing the wire (e.g., at a proximal end of the catheter) to extend the securing mechanism out of the catheter. The securing mechanism can be configured to curve outward toward the coronary sinus wall. As the securing mechanism extends out of the catheter, the distal portion of the securing mechanism contacts the coronary sinus wall to secure the distal end of the catheter in place. The more the mechanically releasing arms are advanced, the closer the approach to the wall and the more force placed against the wall to anchor the catheter in place. The mechanically releasing arms can include a plurality of wires that angle or curve toward the vessel wall when deployed, one or more wires that coil away from the catheter to contact the vessel wall when deployed, or a stopper arm with a curved endcap that contacts the vessel wall when deployed. The securing mechanisms can extend from one side of the catheter or from a plurality of sides of the catheter.

The disclosed securing mechanisms are configured for use with a catheter, such as a guidewire delivery catheter. Disclosed herein are guidewire delivery catheters that have a securing mechanism (or anchor member) that includes one or more mechanically releasing arms. A first example securing mechanism includes two or more wires that advance out of a distal portion of the catheter to curve toward and contact the wall of the coronary sinus. A second example securing mechanism includes a wire that advances out of a distal portion of the catheter and coils toward and contacts the wall of the coronary sinus. A third example securing mechanism includes a wire that is advanced out of a distal portion of the catheter and includes a curved endcap at the end of the wire, the wire angled toward the wall of the coronary sinus.

FIGS. 4A-4D illustrate a first example securing mechanism 400. The securing mechanism 400 includes two or more wires 401 housed along a length of the catheter 22 in a delivery configuration, as shown in FIG. 4A. The two or more wires 401 can be advanced out of a distal portion of the guidewire delivery catheter, at wire ports 403, to press against the wall of the coronary sinus to assume a deployment configuration, as shown in FIG. 4B. The securing mechanism 400 can be guided into place as part of the catheter 22 to where the needle 34 is to puncture the wall of the coronary sinus 30. Once in place, the wires 401 are pushed or advanced so that distal portions 402 of the wires 401 extend out of the distal portion of the catheter 22 and curve away from the catheter 22. Continued advancement of the wires 401 causes a distal portion 402 of each wire 401 to contact the wall of the coronary sinus 30, thereby securing the distal end of the guidewire delivery catheter 22 in place, as shown in FIG. 4C. The wires 401 can extend along a length of the catheter to a proximal portion of the catheter 22 to allow a user to advance the wires 401 from outside an operative site. FIGS. 4A-4C illustrate the wire ports 403 as being positioned distally to the needle port 28. However, it is to be understood that the wire ports 403 can be positioned proximally to the needle port 28 or approximately even with the needle port 28. Similarly, although the wire ports 403 are positioned at a same distal distance, it is to be understood that each wire port 403 can be positioned with different distal distances. The wires 401 can be pulled or withdrawn to retract the wires 401 into the catheter 22 to resume the delivery configuration.

The securing mechanism 400 material can be a compliant, wire material such as a nickel titanium alloy (e.g., Nitinol). In some examples, the distal portions 402 of the wires 401 can include protective material to reduce the likelihood that the distal portions 402 cause damage to the wall 30. In certain examples, the distal portions 402 of the wires 401 are curved to reduce the likelihood that the distal portions 402 cause damage to the wall 30.

FIG. 4C illustrates the wires 401 on opposite sides of the catheter 22 from one another. However, it is to be understood that the wires 401 can be positioned closer together, as shown in FIG. 4D. In addition, FIG. 4C illustrates the wires 401 such that one wire port 403 is on or near the same side of the catheter 22 as the needle port 28. In some instances, this may be undesirable due to the wire 401 on the same side of the needle 34 pushing the catheter 22 away from the wall 30 that is nearest the needle 34. Thus, in some examples, the wires 401 are configured so that the distal portions 402 of the wires push the wall 30 so that the resulting force is in a direction to provide wall apposition for the catheter 22 to push the catheter 22 against the wall 30 nearest the needle 34. In such examples, the wire ports 403 are positioned in a portion of the catheter 22 that is opposite to the portion of the catheter 22 that includes the needle port 28, an example of which is shown in FIG. 4D (which represents a cross-section of the catheter 22). For example, the azimuth angles θ2 and θ3 between the needle port 28 and the wire ports 403 can be at least 90 degrees and less than 180 degrees. It should be understood that reference to the azimuth angle is a reference to cylindrical coordinates, the azimuth angle measured within a cross-section of the catheter, the cross-section forming a surface perpendicular to a longitudinal axis of the catheter. In some examples, the azimuth angles θ2 and θ3 between the needle port 28 and the wire ports 403 can be at least 120 degrees. In some examples, the azimuth angle θ1 between the two wires 402 can be less than 180 degrees, between 0 degrees and 120 degrees, or between 5 degrees and 90 degrees. In some examples, the wire ports 403 are on a radially outer side of the catheter 22 while the needle port 28 is on a radially inner side of the catheter 22. Some example securing mechanisms 400 may include more than two wires and more than two corresponding wire ports. Such wire ports may be distributed on a radially outer side of the catheter 22 and/or may be distributed around the catheter 22.

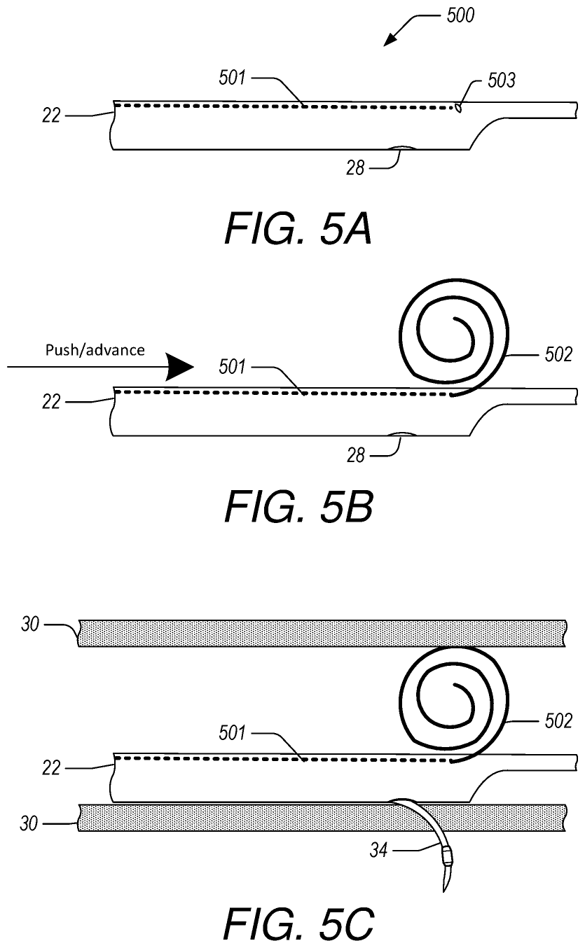

FIGS. 5A-5C illustrate a second example securing mechanism 500. The securing mechanism 500 can be a wire that advances out of a wire port 503 at a distal portion of the guidewire delivery catheter 22. In a delivery configuration, the wire 501 is housed within the catheter 22, as shown in FIG. 5A. As the wire 501 is advanced, a distal portion 502 of the wire 501 exits the wire port 503 and coils to assume a deployment configuration, as shown in FIG. 5B. The coiled distal portion 502 is configured to press against the wall of the coronary sinus 30, as shown in FIG. 5C. The securing mechanism 500 can be guided into place as part of the catheter 22 to where the needle 34 is to puncture the wall of the coronary sinus 30. Once in place, the wire 501 is pushed or advanced (e.g., from a proximal end of the catheter 22) so that a distal portion 502 of the wire 501 extends out of the wire port 503 of the catheter 22 and curves away from the catheter 22. Continued advancement of the wire 501 causes a distal portion 502 of the wire to coil and to contact the wall of the coronary sinus 30, thereby securing the distal end of the guidewire delivery catheter 22 in place. The wire 501 can extend along a length of the catheter to a proximal portion of the catheter 22 to allow a user to advance the wire 501 from outside an operative site. The material of the securing mechanism 500 can be a compliant, wire material such as a nickel titanium alloy (e.g., Nitinol). In some examples, the material can be shape set so that it coils upon exiting the wire port 503 to form the coil portion 502 of the wire 501. The wire 501 can be pulled or withdrawn to retract the wire 501 into the catheter 22 to resume the delivery configuration.

The wire port 503 is illustrated as being positioned distally to the needle port 28. However, it is to be understood that the wire port 503 can be positioned proximally to the needle port 28 or approximately even with the needle port 28. In addition, although a single wire 501 is illustrated, it is to be understood that a plurality of coiling wires can be used, with configurations similar to the securing mechanism 400 described with reference to FIGS. 4A-4D. As in the securing mechanism 400, the wire 501 (or at least the coiled portion 502 of the wire 501) can be coated with a material to reduce the risk of harming the vessel wall that it contacts.

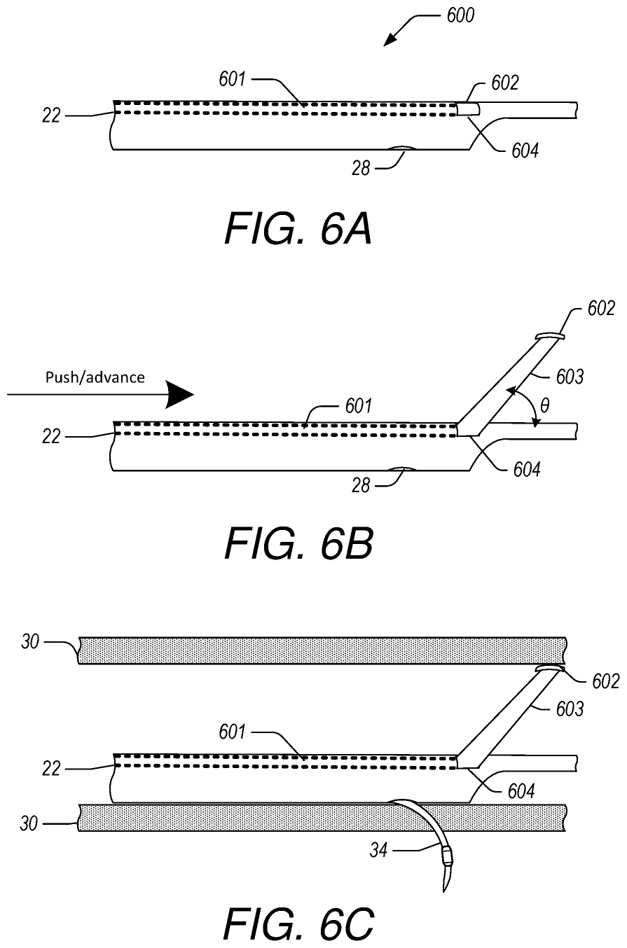

FIGS. 6A-6C illustrate a third example securing mechanism 600. The securing mechanism 600 can be a wire 601 that advances out of wire port 604 in a distal portion of the guidewire delivery catheter 22, as shown in FIG. 6A. The securing mechanism 600 includes a curved endcap 602 at a distal end of the wire 601. The curved endcap 602 acts to cover the wire port 604 in a delivery configuration, as shown in FIG. 6A. As such, the curved endcap 602 can be shaped to approximately follow a curvature of the catheter 22 at the wire port 604. As the wire 601 is advanced to change from the delivery configuration to the deployment configuration, a distal portion 603 of the wire 601 extends at an angle to the catheter 22, as shown in FIG. 6B. The securing mechanism 600 can be guided into place as part of the catheter 22 to where the needle 34 is to puncture the wall of the coronary sinus 30. Once in place, the wire 601 is pushed or advanced (e.g., from a proximal end of the catheter 22) so that a distal portion 603 of the wire 601 extends out of the wire port 604 to extend the curved endcap 602 towards the wall of the coronary sinus 30. Continued advancement of the wire 501 causes the curved endcap 602 to contact the wall of the coronary sinus 30, thereby securing the distal end of the guidewire delivery catheter in place, as shown in FIG. 6C. The curved endcap 602 can be configured to approximately follow the curvature of the wall of the coronary sinus 30. The wire 601 can be pulled or withdrawn to retract the wire 601 into the catheter 22 to resume the delivery configuration. When retracted, the curved endcap 602 can return to cover the wire port 604 to resume the delivery configuration.

The securing mechanism material can include a compliant, wire material such as a nickel titanium alloy (e.g., Nitinol). The wire 601 can be shape set so that it extends at a targeted angle from the catheter 22 as the wire is advanced out of the wire port 604. The angle, θ, the distal end of the wire 603 makes relative to the longitudinal axis of the catheter 22 can be less than or equal to about 90 degrees, and can be at least about 10 degrees and/or less than or equal to about 75 degrees, or at least about 30 degrees and/or less than or equal to about 60 degrees.

In some examples, the curved endcap 602 can include padding made of a material similar to the material of the outer portion of the catheter 22, such as a polyether block amide (e.g., PEBAX®). The curved endcap 602 can be sized to provide a surface area for a larger area of wall apposition.

The wire port 604 is illustrated as being positioned distally to the needle port 28. However, it is to be understood that the wire port 604 can be positioned proximally to the needle port 28 or approximately even with the needle port 28. In addition, although a single wire 601 is illustrated, it is to be understood that a plurality of wires with curved endcaps can be used, with configurations similar to the securing mechanism 400 described with reference to FIGS. 4A-4D.

Each of the securing mechanisms 400, 500, and 600 can include marker bands that are radio opaque to indicate a position of the securing mechanism, as visualized using fluoroscopy. In such examples, the marker bands can be opposite the needle 34 or needle port 28 to ensure that the needle 34 is located in a desirable position prior to extending the needle to puncture the wall 30.

### Additional Examples

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described herein. The structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

Reference herein to “catheters,” “tubes,” “sheaths,” “steerable sheaths,” and/or “steerable catheters” can refer or apply generally to any type of elongate tubular delivery device comprising an inner lumen configured to slidably receive instrumentation, such as for positioning within an atrium or coronary sinus, including for example delivery catheters and/or cannulas.

What is claimed is:

1. A guidewire delivery catheter used to implant a shunt between the coronary sinus and the left atrium, the catheter comprising:
a needle housed within the catheter in a delivery configuration, the needle configured to extend out of the catheter to puncture a vessel wall in a deployment configuration;
a needle port through which the needle extends out of the catheter to transition from the delivery configuration to the deployment configuration;
a securing mechanism housed within the catheter in a delivery configuration, the securing mechanism comprising a compliant wire configured to be advanced out of the catheter in a deployment configuration; and
a wire port through which the compliant wire of the securing mechanism extends out of the catheter to transition from the delivery configuration to the deployment configuration,
wherein a distal portion of the compliant wire of the securing mechanism is configured to coil upon exiting the wire port such that the coiled distal portion of the compliant wire comes into contact with the vessel wall to provide wall apposition for the needle to puncture the vessel wall.

2. The catheter of claim 1, wherein the compliant wire is configured to run along a length of the catheter such that the compliant wire is configured to be advanced by manipulating a component of the securing mechanism from outside of an operative site.

3. The catheter of claim 1, wherein the wire port is positioned opposite the needle port so that the compliant wire exits the catheter on an opposite side from where the needle exits the catheter.

4. The catheter of claim 1, wherein the wire port is positioned more distally along the catheter relative to the needle port.

5. The catheter of claim 1, wherein the wire port is positioned more proximally along the catheter relative to the needle port.

6. A guidewire delivery catheter used to implant a shunt between the coronary sinus and the left atrium, the catheter comprising:
a needle housed within the catheter in a delivery configuration, the needle configured to extend out of the catheter to puncture a vessel wall in a deployment configuration;
a needle port through which the needle extends out of the catheter to transition from the delivery configuration to the deployment configuration;
a securing mechanism housed within the catheter in a delivery configuration, the securing mechanism comprising a first compliant wire and a second compliant wire that are each configured to be advanced out of the catheter in a deployment configuration;
a first wire port through which the first compliant wire of the securing mechanism extends out of the catheter to transition from the delivery configuration to the deployment configuration; and
a second wire port through which the second compliant wire of the securing mechanism extends out of the catheter to transition from the delivery configuration to the deployment configuration,
wherein a distal portion of the first compliant wire and a distal portion of the second compliant wire come into contact with the vessel wall to provide wall apposition for the needle to puncture the vessel wall.

7. The catheter of claim 6, wherein, in a cross-section of the catheter, an azimuth angle between the needle port and each of the two wire ports is at least 90 degrees and less than 180 degrees.

8. The catheter of claim 6, wherein, in a cross-section of the catheter, an azimuth angle between the needle port and each of the two wire ports is at least 120 degrees.

9. The catheter of claim 6, wherein, in a cross-section of the catheter, an azimuth angle between the two wire ports is less than or equal to 120 degrees.

10. The catheter of claim 6, wherein the first compliant wire and the second compliant wire are each configured to run along a length of the catheter such that each of the first compliant wire and the second compliant wire is configured to be advanced by manipulating a component of the securing mechanism from outside of an operative site.

11. A guidewire delivery catheter used to implant a shunt between the coronary sinus and the left atrium, the catheter comprising:
a needle housed within the catheter in a delivery configuration, the needle configured to extend out of the catheter to puncture a vessel wall in a deployment configuration;
a needle port through which the needle extends out of the catheter to transition from the delivery configuration to the deployment configuration;
a securing mechanism housed within the catheter in a delivery configuration, the securing mechanism comprising a compliant wire configured to be advanced out of the catheter in a deployment configuration; and
a wire port through which the compliant wire of the securing mechanism extends out of the catheter to transition from the delivery configuration to the deployment configuration,
wherein the compliant wire includes a curved endcap at a distal end of the compliant wire so that the curved endcap of the compliant wire comes into contact with the vessel wall to provide wall apposition for the needle to puncture the vessel wall.

12. The catheter of claim 11, wherein the compliant wire is shape set to exit the catheter at an exit angle.

13. The catheter of claim 11, wherein the curved endcap is configured to cover the wire port in the delivery configuration.

14. The catheter of claim 11, wherein the compliant wire is configured to be advanced out of the catheter through the wire port by pushing a proximal portion of the compliant wire at a proximal end of the catheter.

15. The catheter of claim 11, wherein the compliant wire is configured to run along a length of the catheter such that the compliant wire is configured to be advanced by manipulating a component of the securing mechanism from outside of an operative site.

16. The catheter of claim 11, wherein the compliant wire comprises a shape memory metal.

17. The catheter of claim 11, wherein the curved endcap is coated with a material to reduce a likelihood of damaging the vessel wall.

18. The catheter of claim 11, wherein the wire port is positioned opposite the needle port so that the compliant wire exits the catheter on an opposite side from where the needle exits the catheter.

19. The catheter of claim 11, wherein the wire port is positioned more distally along the catheter relative to the needle port.

20. The catheter of claim 11, wherein the wire port is positioned more proximally along the catheter relative to the needle port.
